(19) United States
(12) Patent Application Publication
(10) Pub. No.: US 2013/0183395 A1
(43) Pub. Date: Jul. 18, 2013

(54) COMPOSITIONS COMPRISING BOTANICALS, INCLUDING THE USE AND METHOD OF USE THEREOF

(71) Applicant: Krista Coventry, Guelph (CA)

(72) Inventor: Krista Coventry, Guelph (CA)

(73) Assignee: Fem Med Formulas Limited Partnership, Toronto (CA)

(21) Appl. No.: 13/699,529

(22) PCT Filed: May 26, 2010
(86) PCT No.: PCT/CA10/00817

§ 371 Date: Apr. 3, 2013

(51) Int. Cl. A61K 36/79; A61K 31/566; A61K 45/06; A61K 31/59; A61K 36/185; A61K 31/404

(52) U.S. Cl. CPC A61K 36/79; A61K 36/185; A61K 31/404; A61K 45/06; A61K 31/59; A61K 31/566
USPC 424/774

(57) ABSTRACT

A composition comprising indole-3-carbinol, milk thistle, schizandra, stinging nettle, hydroxymatairesinol ligands and optionally calcium-d-glucarate and 400 KJ Vitamin D for altering urinary estrone metabolite and preventing breast cancer in pre and post menopausal women

### FIELD

The present invention relates to compositions comprising botanicals, the use and the method of use thereof, and in particular compositions comprising indole-3-carbinol for altering urinary estrone metabolite levels and preventing breast cancer in women.

### BACKGROUND

As health care costs for disease treatments continue to soar—science, industry, healthcare systems and health care practitioners continue to search for those products which will best serve the preventative model to reduce treatments costs, and provide individuals with healthy and safe products which produce positive health outcomes. According to health statistics from Statistics Canada and the U.S. Department of Health, breast cancer is the most common cancer among women. Over her lifetime, one in nine women will be diagnosed with breast cancer, and one in twenty-seven will die from it. The majority of breast cancers are estrogen-dependent, so maintaining a healthy estrogen balance will reduce breast cancer risk.

Studies have found that two specific metabolites of estrogen metabolism affect breast cancer susceptibility. As urinary levels of 2-hydroxyestrone (2-OHE) increase and levels of 16-αhydroxyestrone decrease (16-αOHE), the risk for breast cancer decreases, because 16-αOHE is an independent risk factor for breast cancer. In most human trials, results are presented as a ratio of urinary 2-OHE to urinary 16-αOHE, and the higher the ratio, the better, in terms of breast health. The optimal urinary ratio of 2-OHE to 16αOHE is 2:1, while a 1:1 ratio is associated with increased breast cancer risk. This ratio is commonly referred to as the estrogen metabolite ratio (EMR).

In a prospective study by Muti et al. (2000), 10,786 Italian women were followed for 5.5 years, and the EMR was measured at baseline in all of these women. The number of diagnosed breast cancer cases that developed during the study period was compared with baseline EMRs. In premenopausal women, those with the higher ratio had, on average, an odds ratio for breast cancer of 0.58, compared to those with a lower ratio. This means that their risk of developing breast cancer was just over half that of the lower ratio group—i.e. the women with a optimal ratio had approximately 50% less risk than those with a sub-optimal ratio. In a case-control U.S. trial (breast cancer cases, compared to matched non-breast cancer controls) published by Kabat et al. (1997) in post-menopausal there was a strong inverse relationship between EMR and breast cancer, and a strong positive relationship between 16-αOHE and breast cancer. This means that the higher the ratio, the lower the breast cancer incidence, and the higher the level of 16-αOHE, the higher the incidence. The authors of a U.K.-based prospective trial reported that post-menopausal women who developed breast cancer over the 8 years of the trial had, on average, a 15% lower EMR than matched controls. Also, women whose ratio was in the highest third had a 30% lower risk of breast cancer development than those in the lower two-thirds EMR (Meilahn et al., 1998).

Indole-3-carbinol (I3C) helps maintain healthy estrogen levels in the body by balancing estrogen metabolites. It does this by shifting the pathway by which estrogens are metabolized. Estradiol undergoes initial oxidative conversion to estrone. Estrone and estradiol can undergo hydroxylation by different cytochrome p450 isoenzymes. Hydroxylation occurs either in the 2 or 16 carbon.

These different metabolites have opposing effects on estrogen receptor-positive breast cancer cells. 16-αOHE stimulates proliferation, while 2-OHE shows no effect on cell growth. Human studies have shown that I3C can shift the balance of estrogen metabolites in the body from 16-αOHE to 2-OHE.

In one human trial, 17 of 20 women (85%) supplemented with 200 or 400 mg/day I3C sustained an average increase of 44% in the ratio of 2-OHE to 16-αOHE over the course of the trial. In another human trial, the 2-OHE:16-αOHE ratio in four out of five obese women who were supplemented with I3C were increased, on average, by 93% over a two-month period. In the same trial, this ratio in non-obese women increased by an average of 38%. In a third human trial, all 20 women who consumed 300 or 400 mg of I3C for a period of 4 weeks significantly increased this ratio. A fourth study reported that in 20 women consuming 400 mg I3C daily, the average increase in the 2-OHE:16-αOHE ratio was 65%. Overall, supplementation with 200-400 mg IC3 appears to increase this critical ratio by approx. 60% in the vast majority of women who consume it on a regular basis.

### SUMMARY OF INVENTION

In one aspect, there is provided a composition for altering urinary estrone metabolite levels, wherein the composition comprises: Indole-3-Carbinol; Milk Thistle (Silybum marianum seed extract, 2:1); Schizandra (Schisandra chinensis fruit extract, 4:1); Stinging Nettle (Urtica dioica leaf extract, 4:1); and Hydroxymatairesinol Lignans (Picea abies [Norway Spruce]).

In another aspect, the composition is provided for preventing breast cancer in a woman.

There is also provided a use of a composition comprising Indole-3-Carbinol, Milk Thistle (Silybum marianum seed extract, 2:1), Schizandra (Schisandra chinensis fruit extract, 4:1), Stinging Nettle (Urtica dioica leaf extract, 4:1), and Hydroxymatairesinol Lignans (Picea abies [Norway Spruce]) for altering urinary estrone levels.

In an alternate aspect, a use of the composition is provided for preventing breast cancer in a woman.

There is further provided a method of use of a composition comprising Indole-3-Carbinol, Milk Thistle (Silybum marianum seed extract, 2:1), Schizandra (Schisandra chinensis fruit extract, 4:1), Stinging Nettle (Urtica dioica leaf extract, 4:1), and Hydroxymatairesinol Lignans (Picea abies [Norway Spruce]) for altering urinary estrone levels wherein a therapeutically effective dose of the composition is administered to a woman twice daily.

In another aspect, a method of use of the composition is provided for preventing breast cancer wherein a therapeutically effective dose of the composition is administered to a woman twice daily.

### DETAILED DESCRIPTION

There is described herein a unique composition that is specifically designed and intended to reduce the risk of breast cancer by increasing and optimizing the EMR.

In one embodiment, there is provided a composition for altering urinary estrone metabolite levels in a woman, wherein the composition comprises: Indole-3-Carbinol; Milk Thistle (Silybum marianum seed extract, 2:1); Schizandra (Schisandra chinensis fruit extract, 4:1); Stinging Nettle (Urtica dioica leaf extract, 4:1); and Hydroxymatairesinol Lignans (Picea abies [Norway Spruce]). In a preferred embodiment, the composition further comprises Vitamin D (Cholecalciferol [Vitamin D3]) and/or Calcium-D-Glucarate.

In a further aspect, the composition for altering urinary estrone metabolite levels in a woman comprises: about 200 mg of Indole-3-Carbinol; about 100 mg of Milk Thistle (Silybum marianum seed extract, 2:1); about 75 mg of Schizandra (Schisandra chinensis fruit extract, 4:1); about 50 mg of Stinging Nettle (Urtica dioica leaf extract, 4:1); and about 10 mg of Hydroxymatairesinol Lignans (Picea abies [Norway Spruce]). Preferably, the composition further comprises about 400 IU/10 mcg of Vitamin D (Cholecalciferol [Vitamin D3]) and/or about 75 mg of Calcium-D-Glucarate.

In another embodiment, there is provided a composition for preventing breast cancer in a woman, wherein the composition comprises: Indole-3-Carbinol; Milk Thistle (Silybum marianum seed extract, 2:1); Schizandra (Schisandra chinensis fruit extract, 4:1); Stinging Nettle (Urtica dioica leaf extract, 4:1); and Hydroxymatairesinol Lignans (Picea abies [Norway Spruce]). In a preferred embodiment, the composition further comprises Vitamin D (Cholecalciferol [Vitamin D3]) and/or Calcium-D-Glucarate.

In a further aspect, the composition for preventing breast cancer in a woman comprises: about 200 mg of Indole-3-Carbinol; about 100 mg of Milk Thistle (Silybum marianum seed extract, 2:1); about 75 mg of Schizandra (Schisandra chinensis fruit extract, 4:1); about 50 mg of Stinging Nettle (Urtica dioica leaf extract, 4:1); and about 10 mg of Hydroxymatairesinol Lignans (Picea abies [Norway Spruce]). Preferably, the composition further comprises about 400 IU/10 mcg of Vitamin D (Cholecalciferol [Vitamin D3]) and/or about 75 mg of Calcium-D-Glucarate.

In another embodiment, the use of a composition comprising Indole-3-Carbinol, Milk Thistle (Silybum marianum seed extract, 2:1), Schizandra (Schisandra chinensis fruit extract, 4:1), Stinging Nettle (Urtica dioica leaf extract, 4:1), and Hydroxymatairesinol Lignans (Picea abies [Norway Spruce]) is provided for altering urinary estrone levels. In preferred embodiments the composition further comprises Vitamin D (Cholecalciferol [Vitamin D3]) and/or Calcium-D-Glucarate.

The use of a composition for altering urinary estrone levels increases the ratio of 2-hydroxyestrone relative to 16-αhydroxyestrone. Preferably, the ratio of 2-hydroxyestrone relative to 16-αhydroxyestrone is about 2:1.

In another embodiment, a composition comprising Indole-3-Carbinol, Milk Thistle (Silybum marianum seed extract, 2:1), Schizandra (Schisandra chinensis fruit extract, 4:1), Stinging Nettle (Urtica dioica leaf extract, 4:1), and Hydroxymatairesinol Lignans (Picea abies [Norway Spruce]) is provided for use in preventing breast cancer in a woman. In preferred embodiments the composition further comprises about 400 IU/10 mcg of Vitamin D (Cholecalciferol [Vitamin D3]) and/or about 75 mg of Calcium-D-Glucarate.

The use of the composition for preventing breast cancer is preferably administered to both pre- or post-menopausal women.

In another embodiment, a method is provided for altering urinary estrone metabolite levels in a woman comprising administering to the woman a therapeutically effective amount of a composition comprising Indole-3-Carbinol, Milk Thistle (Silybum marianum seed extract, 2:1), Schizandra (Schisandra chinensis fruit extract, 4:1), Stinging Nettle (Urtica dioica leaf extract, 4:1), and Hydroxymatairesinol Lignans (Picea abies [Norway Spruce]) twice a day. In a preferred embodiment, the composition further comprises Vitamin D (Cholecalciferol [Vitamin D3]) and/or Calcium-D-Glucarate.

In some embodiments, a method is provided for preventing breast cancer in a woman comprising administering to the woman a therapeutically effective amount of a composition comprising Indole-3-Carbinol, Milk Thistle (Silybum marianum seed extract, 2:1), Schizandra (Schisandra chinensis fruit extract, 4:1), Stinging Nettle (Urtica dioica leaf extract, 4:1), and Hydroxymatairesinol Lignans (Picea abies [Norway Spruce]) twice a day. In a preferred embodiment, the composition further comprises Vitamin D (Cholecalciferol [Vitamin D3]) and/or Calcium-D-Glucarate.

Advantageously, the use of a composition of the present invention may also have clinical utility as a preventative tool. Clinicians such as General Practitioners, Naturopathic Doctors and Registered Dieticians may use a composition of the present invention to conduct a ‘Measure-Treat-Measure’ program using a composition of the present invention as the ‘Treat’ and urinary estrogen metabolite testing as the ‘Measure’ to capture the risk reduction factors for a woman's preventative breast health. This risk reduction factor is captured within about 30 days of supplementation based on the biomarker, the treatment influences and the risk reduction associated with the improvement of the biomarker.

The following example is illustrative of various aspects of the invention, and does not limit the broad aspects of the invention as disclosed herein.

### EXAMPLE

### Example 1

A double blind placebo controlled parallel clinical trial has been conducted to demonstrate the safety, efficacy and positive health outcomes of an embodiment of the present invention consisting of five botanicals including indole-3-carbinol, Schisandra chinesis, Milk thistle, Stinging nettle, and HMR lignans, in addition to vitamin D and calcium-D-glucarate for the alteration of urinary estrone metabolite levels in both pre- and post-menopausal women, with or without Hormone Replacement Therapy.

### Pilot Trial Results

Preliminary reports from the clinical trial investigating a composition of the present invention resulted in significant improvement in the EMR. Both pre- and post-menopausal women using the composition had similar effects compared to baseline and to the placebo, showing that the product exhibits positive outcomes and risk reduction factors across the demographic spectrum of women's breast health.

Stage One findings on a combined group of pre- and post-menopausal women from the clinical trial investigation has demonstrated statistical significance in these women as a combined group. This statistical significance demonstrates that the composition of the invention is capable of bringing women into the optimal ratio for breast cancer prevention purposes within 30 days and will allow for optimal publication potential.

In addition to the positive outcomes recorded in the pilot trial and completion of Stage One, trial participants had no serious adverse events.

### Time-Lines

The trial commenced on Day 0, and recruitment of the first 25 subjects (pre- and post-menopausal women not taking hormones) was completed by Day 18. Completion of sample collection for the 25th recruited subject took place on Day 50. Blood and urine samples were delivered to the testing lab by Day 53, and analysis of blood and urine samples took place over the following 4 weeks. Results were received on Day 80.

On Day 228, a second batch of blood and urine samples were tested. This batch included pre-menopausal women from subject #20 to subject #40, and post-menopausal women from subject #8 to subject #28. The urinary estrogen metabolite results from this group are now available. These results have been combined with the results from the previous group of subjects.

### Results

In an analysis of the whole data (i.e. pre- and post-menopausal data combined), there was a statistically significant difference in the 2-OHE:16-OHE ratio between the supplement group and the placebo group.

The urinary estrone metabolite data for pre- and post-menopausal women were analysed separately. Because some of the estrogen metabolites of interest were either absent from the urine, or present at undetectable levels (see previous progress report), data for 4 pre-menopausal women (subjects 08, 09, 018 and 019) and 8 post-menopausal women (subjects 01, 02, 05, 06, 07, 12, 013 and 014) was omitted. Therefore, data from 36 pre-menopausal women and 19 post-menopausal women were included for assessment. Because of the nature of the randomisation process, in which randomisation for all 144 subjects was completed, an even number of subjects in each sub-group does not occur until all subjects have been recruited and the trial is completed. Thus, there were 17 pre-menopausal women consuming the placebo and 19 consuming the treatment. In the post-menopausal group, 10 women consumed the placebo and 9 consumed the treatment.

A total sample size of 55 subjects is sufficient to detect a difference, if one is present, and this beneficial difference in the 2:16OHE ratio (the biomarker of greatest interest), in the supplement group was observed when both pre- and post-menopausal subjects' data was combined.

Analysis of enterolactone levels for the second batch of samples are being analysed and will be assessed as part of a subsequent report.

TABLE 1
2-hydroxyestrone levels, 16-hydroxyestrone levels and calculated 2-OH estrone:16-OH
estrone levels in urine of women consuming the composition or a placebo (average values and standard deviation).

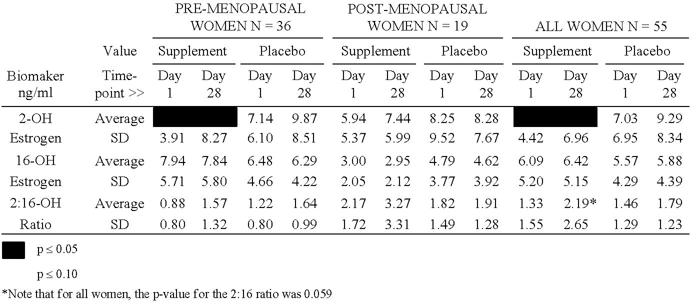

The change in 2-OHE levels in subjects consuming the supplement is quite remarkable. In the pre-menopausal group, 2-OHE levels more than doubled (4.68 to 9.43), while the placebo group increased 2-OHE levels by just over one-third (7.14 to 9.87). The ratio of 2-OHE to 16-OHE showed a very similar pattern. For pre-menopausal women, the composition of the present invention appears to be increasing the 2:16 hydroxyestrone ratio much more than the placebo, i.e. the composition of the present invention almost doubled the ratio from an average of 0.88 to an average of 1.57 during the 28-day supplementation, whereas in the placebo group, this ratio rose by only one-third, from 1.22 to 1.57.

For post-menopausal women, 2-OHE levels increased by 25% (5.94 to 7.44), but did not change in the placebo group. The composition of the present invention appears to increase the 2:16 hydroxyestrone ratio in a similar manner to that of the pre-menopausal women, i.e. the composition of the present invention increased the ratio by 50%, from an average of 2.17 to an average of 3.27 during the 28-day supplementation, whereas in the placebo group, this ratio rose by only 5%, from 1.82 to 1.91. As stated previously, statistical analysis of the data (i.e. pre- and post-menopausal data combined), revealed a statistically significant difference in the 2-OHE:16-OHE ratio between the supplement group and the placebo group.

Note that the change in ratio for both the pre- and post-menopausal women consuming the supplement was almost entirely due to a significant increase in the 2-OHE levels. There was virtually no change in the 16-OHE levels.

### Conclusions

From this data, and particularly from the statistically significant result of a beneficial change in the 2:16OHE ratio in the supplement group, the composition of the present invention has had a beneficial effect on the ratio of 2-hydroxyestrone to 16-hydroxyestrone, which would indicate, based on prior literature, a reduction in breast cancer risk.

Although preferred embodiments of the invention have been described herein, it will be understood by those skilled in the art that variations may be made thereto without departing from the spirit of the invention or the scope of the appended claims. All references mentioned herein are incorporated by reference in their entirety.

### REFERENCES

Bell, M., Crowley-Nowick, P., Bradlow, H., Sepkovic, D., Schmidt-Grimminger, D., et al. 2000 Placebo-controlled trial of Indole-3-carbinol in the treatment of CIN. Gynecologic Oncology 78: 123-129.
Bradlow, H., Michnovicz, J., Halper, M., Miller, D., Wong, G. and Osborne, M. 1994 Long-term responses of women to indole-3-carbinol or a high fiber diet. Cancer Epidemiology, Biomarkers and Prevention. 3: 591-595.
Kabat G., Chang, C., Sparano, J., Sepkovic, D., Khalil, A. Et al. 1997 Urinary estrogen metabolites and breast cancer: a case-control study. Cancer Epidemiol Biomarkers Prev. 6(7): 505-509.
Meilahn, E., De Stavola, B., Allen, D., Fentiman, I., Bradlow, H. et al. 1998 Do urinary oestrogen metabolites predict breast cancer? Guernsey III cohort follow-up. Br. J. Cancer 78(9): 1250-1255.
Michnovicz, J. 1998 Increased estrogen 2-hydroxylation in obese women using oral indole-3-carbinol. International Journal of Obesity 22: 227-229.

Muti, P., Bradlow, H., Micheli, A., Krogh, V., Freudenheim, J. et al. 2000 Estrogen metabolism and risk of breast cancer: a prospective study of the 2:16alpha-hydroxyestrone ratio in premenopausal and postmenopausal women. Epidemiology 11(16): 635-640.

Wong, G., Bradlow, L., Sepkovic, D., Mehl, S and Mailman, J. 1997 Dose-ranging study of indole-3-carbinol for breast cancer prevention. Journal of cellular biochemistry supplements 28/29: 111-116.

1. A composition comprising: Indole-3-Carbinol; Milk Thistle; Schizandra; Stinging Nettle; and Hydroxymatairesinol Lignans.

2. The composition of claim 1 further comprising Vitamin D (Chloecalciferol [Vitamin D3]).

3. The composition of claim 1 further comprising Calcium-D-Glucarate.

4. The composition according to claim 1 comprising:
about 200 mg of Indole-3-Carbinol;
about 100 mg of Milk Thistle (Silybum marianum seed extract, 2:1);
about 75 mg of Schizandra (Schisandra chinensis fruit extract, 4:1);
about 50 mg of Stinging Nettle (Urtica dioica leaf extract, 4:1); and
about 10 mg of Hydroxymatairesinol Lignans (Picea abies [Norway Spruce]).

5. The composition according to claim 4 further comprising about 400 IU/10 mcg of Vitamin D (Cholecalciferol [Vitamin D3]).

6. The composition according to claims 4 further comprising about 75 mg of Calcium-D-Glucarate.

7. A composition consisting of: Indole-3-Carbinol; Milk Thistle; Schizandra; Stinging Nettle; and Hydroxymatairesinol Lignans.

8. The composition according to claim 7 consisting of:
about 200 mg of Indole-3-Carbinol;
about 100 mg of Milk Thistle (Silybum marianum seed extract, 2:1);
about 75 mg of Schizandra (Schisandra chinensis fruit extract, 4:1);
about 50 mg of Stinging Nettle (Urtica dioica leaf extract, 4:1); and
about 10 mg of Hydroxymatairesinol Lignans (Picea abies [Norway Spruce]).

9. A composition for altering urinary estrone metabolite levels in a woman, wherein the composition consists of:
200 mg of Indole-3-Carbinol;
100 mg of Milk Thistle (Silybum marianum seed extract, 2:1);
75 mg of Schizandra (Schisandra chinensis fruit extract, 4:1);
50 mg of Stinging Nettle (Urtica dioica leaf extract, 4:1);
10 mg of Hydroxymatairesinol Lignans (Picea abies [Norway Spruce]);
400 IU/10 mcg of Vitamin D (Cholecalciferol [Vitamin D3]); and
75 mg of Calcium-D-Glucarate
as medicinal ingredients.

10. The composition of claim 9 further comprising cellulose, water, microcrystalline cellulose, and magnesium stearate as non-medicinal ingredients.

11.-20. (canceled)

21. A method for altering urinary estrone metabolite levels in a woman, comprising administering to said woman a therapeutically effective amount of the composition of claim 1.

22. The method of claim 21 wherein the therapeutically effective amount of the composition is administered two times daily.

23. A method for preventing breast cancer in a woman, comprising administering to said woman a therapeutically effective amount of the composition of claim 1.

24. The method of claim 23 wherein the therapeutically effective amount of the composition is administered two times daily.
